(19) United States
(12) Patent Application Publication
(10) Pub. No.: US 2011/0042215 A1
(43) Pub. Date: Feb. 24, 2011

(54) AC FIELD INDUCED BIOMOLECULE CYRSTALLIZATION AND HYDRATION CAGE DISRUPTION

(76) Inventors: Diana Hou, Lansdale, PA (US); Hsueh-Chia Chang, Granger, IN (US)

Correspondence Address: LADAS & PARRY LLP, 224 SOUTH MICHIGAN AVENUE, SUITE 1600, CHICAGO, IL, 60604, US

(21) Appl. No.: 12/867,597

(22) PCT Filed: Feb. 13, 2009
(86) PCT No.: PCT/US09/34094

§ 371 Date: Nov. 4, 2010

Related U.S. Application Data
(60) Provisional application No. 61/065,742, filed on Feb. 14, 2008.

(51) Int. Cl. C30B 7/00; C07K 1/26

(52) U.S. Cl. 204/547; 204/643

(57) ABSTRACT

An apparatus and methods for biomolecular crystallization is disclosed. The method includes providing biomolecule solution and bringing the biomolecule solution into direct contact with a plurality of electrodes. An alternating current is applied to the plurality of electrodes to impart a dielectrophoresis force upon the biomolecule solution and to form at least one crystal from the biomolecule solution.

### CROSS REFERENCE TO RELATED APPLICATION

This application is a non-provisional application claiming priority from U.S. Provisional Application Ser. No. 61/065,742, filed Feb. 14, 2008, and incorporated herein by reference in its entirety.

### FIELD OF THE DISCLOSURE

The present disclosure relates generally to biomolecule crystallization and more particularly to AC field induced biomolecule crystallization and hydration cage disruption.

### BACKGROUND OF RELATED ART

Breakthroughs in genetic engineering and drug design have led the need to determine biological macromolecular structures in order to create more effective drugs. Knowing the structures of these biomolecules, such as, for example, protein molecules, carbohydrates, lipids, nucleic acids, etc, have led to advances in medical research in structure guided drug design that targets specific protein sites, controlled drug delivery, and genetic engineering of protein drugs. By understanding their native confirmation, the positioning and the activity of specific amino acid groups can be determined so that drugs can be designed to target these specific areas.

The amino acids that constitute the protein molecules have both hydrophobic and hydrophilic functional groups. Therefore the native conformation of the protein molecules is one that will pack the hydrophobic groups on the interior, exposing the hydrophilic groups to the solvent. Because these molecules are very large, ranging from 100 Daltons to 10K Daltons in comparison to other complex molecules and conventional macromolecules, which are roughly 100 Daltons, the protein molecules can form multiple minimal energy state conformations. These conformations are sensitive to the solvent environment such as the pH, temperature, and precipitant concentration, and thus varying conditions can form various protein conformations. Hence it is oftentimes difficult to form protein crystals in their native state in a biological system.

Along with the conformational difficulties the protein molecule poses to crystallization, is hydration of the hydrophilic groups on the exterior of the protein structure. In solution, these hydrophilic groups can form hydrogen bonds with the water molecules to neutralize the charges of the protein molecule, driving the solubility of the protein in water but hindering the crystallization process. In solvent, the water can form structured shells of water molecules surrounding the protein molecules, a “hydration cage.” These water molecules will act as a dielectric between the macromolecules in solution that shield the protein molecules from the electrostatic influence from one another, hindering and reducing their attraction to each other and their interactions. This “hydration cage” has been found to be a problem in crystallization and mass spectroscopy for proteins and other biological macromolecules such as DNA because of the neutralizing and the shielding effects of the water molecules.

Therefore, the methods used to coerce crystallization rely on slowly altering environmental conditions to allow the molecules to diffuse through the solution and interact with each other.

### SUMMARY

Embedded microelectrodes are fabricated on a substrate such that a biomolecular solution, such as, for example a protein solution, is in direct contact with the electrodes. An alternating current is applied across the internal electrodes to produce a dielectrophoresis force. In some examples, the AC current also generates a field gradient within the solution. When, for example, protein molecules are in solution, a hydration cage solvates the charged functional groups of amino acids, forming ordered water molecules that surround the entire protein molecule in solution. The AC current is frequency optimized to disrupt the hydration cage surrounding the molecules in solution, allowing the molecules to interact without relying on evaporation and diffusion forces.

In addition to frequency tuning the field, the voltage of the field supplies additional energy into the crystallization system and to the water molecules. With a higher voltage a stronger dipole and polarization effect can occur, forming additional separation of the water molecules and the protein molecules.

### BRIEF DESCRIPTION OF THE DRAWINGS

FIG. 1 is an example protein crystallization phase diagram.

FIG. 2 is an enlarged plan view of an example electrode used in an example AC field enhanced biomolecular crystallization process.

FIG. 3 is an enlarged plan view of another example electrode used in the example AC field enhanced biomolecular crystallization process.

FIG. 4 is a graph showing crystallization of an example biomolecule solution under various frequencies and voltages.

FIG. 5A is a graph of an average size of an example fused crystal produced in the example of FIG. 4.

FIG. 5B is a graph of an average size of an example single crystal produced in the example of FIG. 4

FIG. 6 is an illustration of crystals formed in a control example of FIGS. 5A and 5B.

FIG. 7 is an illustration of a crystal formed in another example crystallization process.

FIG. 8 is a diffraction pattern formed by one example crystal produced in the example crystallization process.

### DETAILED DESCRIPTION

The following description of the disclosed examples is not intended to limit the scope of the invention to the precise form or forms detailed herein. Instead the following disclosure is intended to be illustrative of the principles of the invention so that others may follow its teachings.

As noted above, biomolecular crystals, such as, for example, protein crystals and their structures are of importance for structure-guided drug design and controlled drug delivery. In solution, the solvated molecules are surrounded by a hydration cage. This hydration cage hinders interactions between the protein molecules by acting as a dielectric and shielding the electrostatic attraction between adjacent molecules. Crystallization can typically occur only upon the removal of these hydration cages in supersaturated protein solutions. If the hydration cage is removed too quickly (e.g., by evaporation or an external field), the molecules will not reach their native conformation and the solution will form a gel instead. Alternatively, the solution may enter the spinodal decomposition region where spontaneous nucleation occurs and a massive number of crystals are produced. Therefore, a delicate balance between gelation and spontaneous crystallization is necessary.

Current methods for growing crystals rely on protein desolvation without triggering gel formation or massive nucleation. The diffusion coefficient for lysozyme, for example, during desolvation is estimated to be a low 10⁻⁶ cm²/s. Existing methods such as vapor diffusion, seeding, microfluidics, electric field, and magnetic fields have attempted to accelerate diffusion and increase protein saturation in the solution to enhance crystal nucleation and growth.

For example, direct current (DC) electric fields have been used to orient the molecular dipoles and desolvate the protein molecules. However, when a DC field is applied to internal electrodes in a highly conductive solution, Faradaic reactions occur on the electrodes. To limit the Faradaic reactions, the applied fields are limited to low voltages and low currents, typically no more than 1 V and 20 μA. External electrodes are not limited by Faradaic reactions, but require higher electric fields, typically of up to 6 kV/cm.

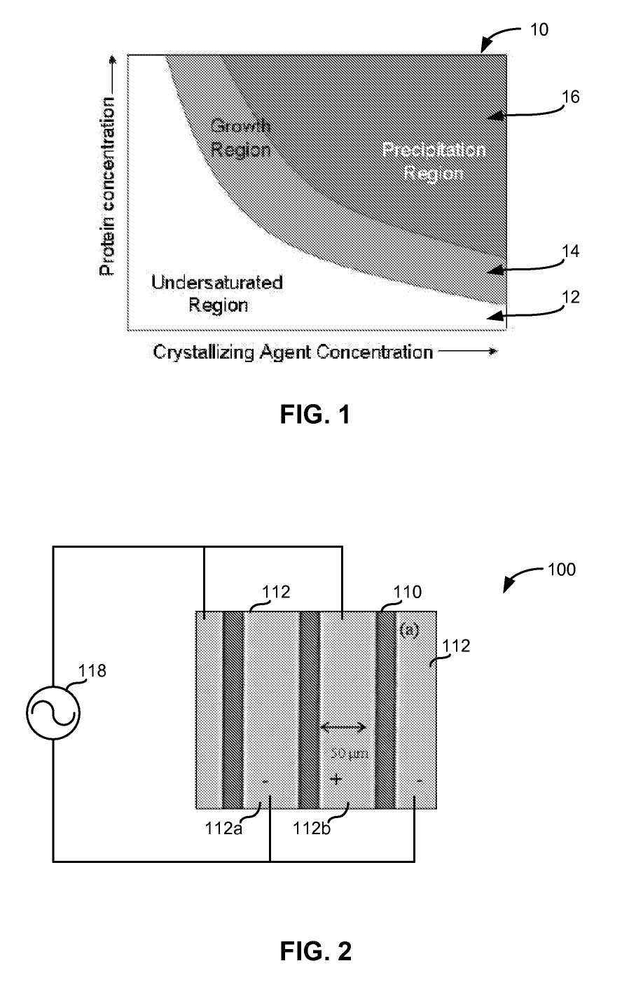

FIG. 1 illustrates a protein crystallization phase diagram 10 that illustrates the probability of crystals growth and crystal nucleation as a function of some additive precipitant concentration. Three regions are defined based on the saturation limits of the protein in solution. In an undersaturated region 12, the protein molecules are soluble in solution and crystals will dissolve back into solution. In a metastable zone 14, the region above the undersaturated region 12, the protein solution is saturated such that proteins will not spontaneous nucleate out of solution but is saturated such that already existing crystals can grow. In a labile region 16, the protein concentration is supersaturated and the molecules can spontaneously nucleate out of solution.

Protein crystallization, however, is not as simple as designing a path to enter and exit the labile region 16. The problem lies in the rate of nucleation and the rate of growth of the crystals in solution. Within the labile region 16, nucleation can occur rapidly such that many small crystals form, which will quickly bring the solution from the labile region 16 to the metastable region 14. However, these crystals are generally small and due to the large number of individual crystals, the size of the crystals will be limited. Ideally, a path enters the labile region 16 only for a short time such that a small of crystals nucleate and cause the path to drop into the metastable region 14. However this path is difficult to control to such precision, thus driving multiple crystallization techniques that slowly change the environment conditions in order to follow the desired path.

The majority of previous crystallization techniques rely on diffusion of the protein molecules and the water molecules. The most widely used method is vapor diffusion which can be used in a hanging drop or a sitting drop apparatus. Because of the small amount of liquid used and its simple apparatus it is mostly used for screening a broad spectrum of crystallization conditions. The driving mechanism in vapor diffusion is the drop of protein solution that will slowly concentrate due to the diffusion of the water in the reservoir. In the sealed system, the diffusion is slow in order to reach equilibrium with the reservoir which is driving the evaporation, thus allowing for the diffusion of the water from the protein solution to the buffer and allowing the solution to slowly enter the labile region 16.

Another well established method is the batch method. Here, an undersaturated protein solution is mixed with a precipitant solution that will alter the protein solubility in the solution immediately creating a supersaturated solution with respect to the protein. Though this method has high yields, it is most often used in single laboratory tests than in broad crystallization spectrums because of the problem with rapid screening that the drop method allows.

Still another method is to use a free interface based on liquid-liquid diffusion. In this technique, the driving mechanism is the equilibrium of a precipitant gradient of solutions that are juxtaposed. These gradients create areas of local supersaturation near the interface of the two liquids and promote the appearance of crystal nuclei.

Still other methods for crystallization involve the use of microfluidic and electrokinetics to manipulate drops and small volumes on a chip to drive rapid crystallization. Some of these methods have included using a free interface diffusion to aide in rapid crystallization, while others have developed mixing chips to determine precipitation diagrams have grown crystals in capillaries for on-chip X-Ray Diffraction (XRD) analysis, and have developed batch reactors on a chip. However, these methods still rely on diffusion based mechanisms to drive the nucleation and crystal growth processes. By limiting the volume, the diffusion length in the system is reduced but is still a slow process.

Another approach to enhancing crystallization is to replace or reduce the diffusion time scale associated with the protein molecules in solution by using microgravity and magnetic and electric fields. Microgravity, either simulated using magnets or through experiments performed in microgravity, reduces the turbulent driven flow in the solution. These flows have been shown to increase the time of crystallization along with limiting the size of the crystal by reducing the depletion layer surrounding the larger protein crystals. However, microgravity experimental set ups are difficult to acquire and the magnetic fields used often required strengths in excess of 10 Tesla. Magnetic fields alone have been proven to coerce crystals to orient themselves in the direction of the magnetic field, however the crystallization requires large fields, up to 10 Tesla magnets.

In keeping with the teachings of the present invention, an AC electric field is used to influence the rate and quality of biomolecule crystallization, and more specifically, protein crystallization. In particular, due to the nature of the alternating current, high fields can be applied to internal electrodes without the consequences of Faradaic reactions of the electrodes and/or ion or bubble contamination. The AC field affects not only nucleation but also affect the quality of the crystal grown. The use of an AC field in addition to the use of internal electrodes causes dielectrophoresis (DEP) forces to manipulate protein molecules and concentrated packets of proteins suspended in solution. Dielectrophoresis is one mechanism used in sorting and concentrating bioparticles ranging from cells to viruses in a microfluidic system. Additionally, the use of an optimum frequency in the AC field for crystallization is possible, as well as the breaking of the hydration cages surrounding the protein molecules in solution.

Turning to FIG. 2, there is illustrated one example electrode 100 used in the crystallization methods and systems described herein. As shown in FIG. 2, the example electrode 100 includes a substrate 110, and a plurality of electrodes 112. The example electrodes 112 are generally straight and, in this example, are arranged parallel to one another. The example substrate 100 is, for example, a glass slide having the electrodes 112 formed using standard lithography techniques. The example electrodes 112 are Ti/Au of a thickness of approximately 5 nm to approximately 25 nm. Additionally, the electrodes 112 have a width of approximately 50 μm and a gap between each electrode 112 of approximately 50 μm. An AC power supply 118 is coupled to the electrode 100 such that some of the electrodes 112a are negatively charged, while adjacent electrodes 112b are positively charged. The AC power supply 118 may be any suitable power supply, including, for example, a sinusoidal AC signal generator such as, for instance, an AGILENT® 33220A, a TEKTRONIC® CFG253, or a HEWLETT PACKARD® 3312A.

Referring to FIG. 3, there is illustrated another example electrode 120 which may also be used in the crystallization methods and systems described herein. Similar to the electrode 100, the electrode 120 includes a substrate 122, and a plurality of electrodes 124 arranged in a multipole fashion, such as, for example, a quadrapole. The example substrate 120 is, similarly, a glass slide having the four electrodes 124 of Ti/Au formed thereon by standard lithography. Each example electrode 124 has a thickness of approximately 5 nm to approximately 25 nm. Additionally, in this example, the four electrodes 124 all have a half angle of 40 degrees to accentuate the difference in gap distance radially from the center. In particular, the gap at the center of the quadrapole measures approximately 10 μm from the opposing tip, while the gap at a distance further radially from the center is approximately 20 μm from the opposing tip. An AC power supply 128 is coupled to the electrode 120 such that alternate electrodes 124a are negatively charged, and adjacent electrodes 124b are positively charged.

In operation, each of the electrodes 100, 120, are brought into direct contact with a biomolecular solution, such as, for instance, a protein solution, to form an internal electrode crystallization system. In one example, the protein solution is Lysozyme as described herein below. An alternating current is applied to the electrodes to impart a dielectrophoresis force upon the solution. The example AC signal is a high frequency, high voltage signal that disrupts the hydration cage of the protein solution, and allows for the formation of at least one crystal. The AC signal may be manipulated as desired, including for example, in the frequency domain and/or the voltage domain. In particular, in one example, the frequency may be varied between approximately 330 KHz and approximately 18 MHz. Similarly, in one example, the voltage may be varied between approximately 19 Vpp and 21 Vpp. Finally, the time which the AC signal is varied may be predetermined and may range anywhere from a very short amount of time to the entire crystallization process.

For instance, in one example method of AC field induced crystallization and hydration cage disruption utilizing the electrodes 100 and 120, a 0.1 M sodium-acetate-acetic buffer was made by mixing 2 ml of 0.1 M Acetic Acid with 3 mL of 0.1 sodium acetate and 5 ml of deionized water to reach a pH of 4.8. Lysozyme (Sigma) was mixed with the buffer at a concentration of 40 mg/mL. A second solution, i.e., the precipitant solution, of 8% w/v sodium chloride was made with the same buffer. Equal mixtures of both, 1 mL of each, were mixed and used in all crystallization procedures.

A sinusoidal AC signal was generated by the power supplies 118, 128 using three different function waveform generators to increase the range of frequencies and voltages available and to alter the current applied in each system. The output and the current in each setup were measured using an oscilloscope, such as for example a TEKTRONIX® TDS 2014, and multimeter probe, such as, for example, a RADIOSHACK® Digital Multimeter. Images were captured using a camera, such as, for example, an OLYMPUS® 1X71 microscope and an I-Speed CDU camera system.

In the example method, a silicon isolation chamber well with an open surface interface and an adhesive backing were used as loading wells holding a volume of 130 μL. The wells were sealed to the substrates 110, 122 with the electrodes 112, 124 centered in the wells. After the addition of the protein solution, the wells were sealed with Parafilm, a coverslip and silicon grease to limit the rate of evaporation from the wells. Controls were set up in a similar fashion and had both electrodes that were not connected to the power supply and a clean glass slide. All samples were crystallized at room temperature.

For analysis, the formed crystals were either stained with an Izit Crystal Dye or used for X-ray diffraction (XRD) analysis. Additionally, a Bruker SMART APEX Diffractometer was used to obtain the diffraction patterns of the crystals which were mounted on a nylon loop and coated to protect the crystal in the liquid nitrogen.

In a first example, the electrode 100 was utilized to illustrate the effect of the frequency of the AC field on the crystal quality. In this example, two control wells with no applied electric field were used along with three wells with an applied electric field of 19.6 Vpp, 18.05 MHz with a current of 16 uA, 21.2Vpp, 2.948 MHz, and a current of 1 mA, and one at 21.4 Vpp, 334 KHz at a current of 391 uA. The wells each contained 130 uL of protein and precipitant solution mixture and were monitored over 4½ days to study their nucleation rate, quality of crystal formed and the size of the crystals formed. A high quality crystal is considered to be one that forms as a single crystal and has no observable defects such as cracks or has plane shifts from forming with adjacent crystals. A low quality crystal is one that has these defects that would be make it unusable for structure analysis.

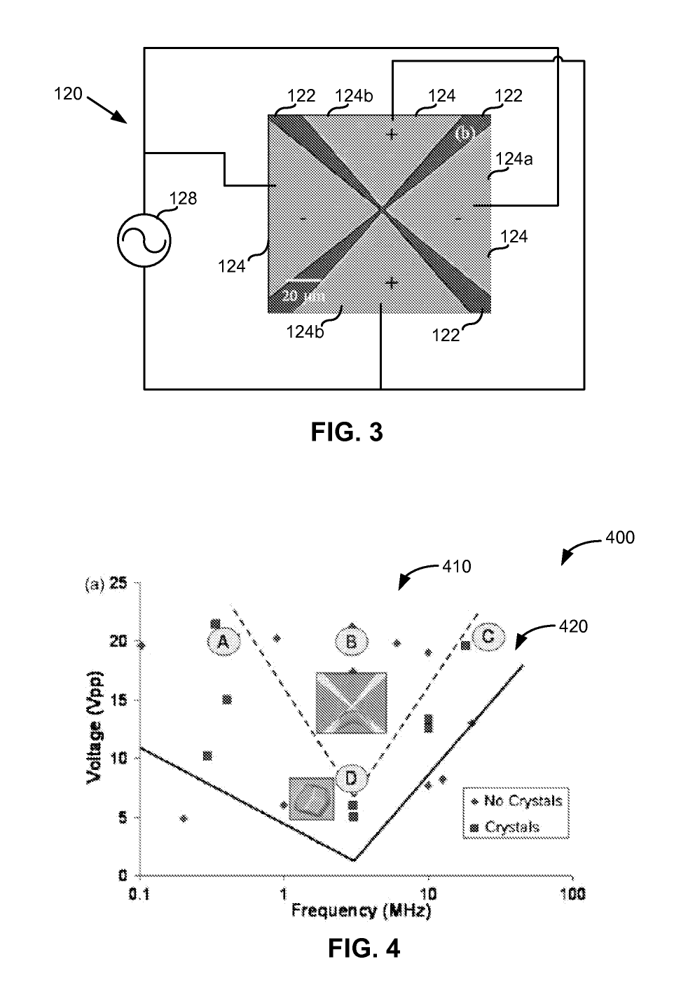

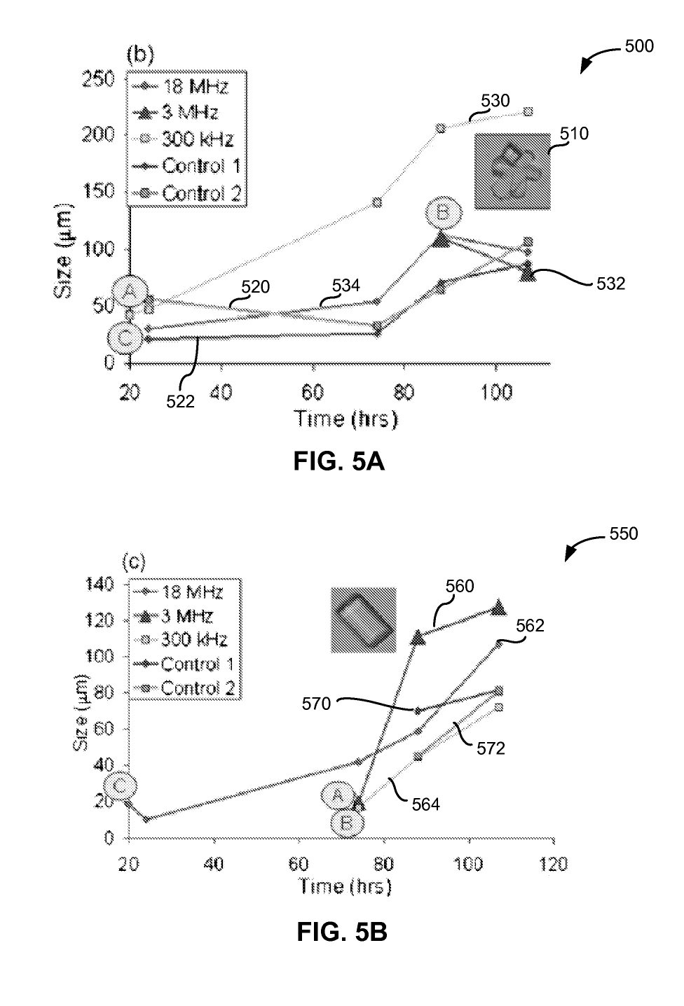

Turning to FIGS. 4 and 5A, 5B, there are illustrated graphs showing the results of the above described crystallization processes. In particular, FIG. 4 is a graph 400 illustrating the example lysozyme solution under different alternating current fields after maturing for 24 hours. The graph 400 illustrates indications of nucleation, where crystals were observed, a square is depicted. Otherwise, if no nucleation was observed, a diamond is depicted. FIG. 5A illustrates a graph 500 showing the average size of the crystals formed over 110 hours, with the alternating current field being active for only the first 24 hours. FIG. 5B illustrates a graph 550 showing the average size of single crystals formed over 110 hours, with an applied field being active for only the first 24 hours.

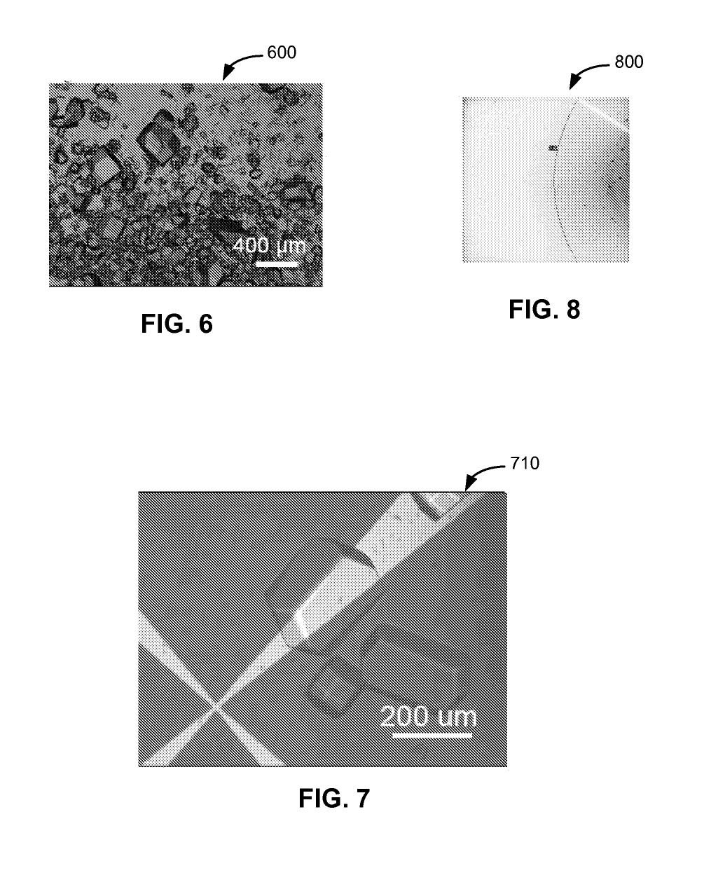

During the example process, both single and fused crystals were seen over the length of the trial and their sizes over time are shown in the graphs 500, 550 in FIGS. 5A, 5B. The fused crystals are illustrated in the graph 500 (FIG. 5A). The largest crystal 510 produced, was measured to be roughly 250 μm. The example crystal 510 was formed in the low frequency limit. In comparison to the controls (lines 520, 522), the other three wells (lines 530, 532, 534), all had fused crystals roughly the same size. An image of the control fused crystals 600 is seen in the inset in FIG. 6. As illustrated in FIG. 6, the formed crystals 600 are not as disjointed as the crystals formed in the low frequency unit, but nevertheless it is still unusable for structure analysis. Additionally, the sample under the mid frequency range (line 532) showed no fused crystals until much later in time. In comparison with the single crystal graph (FIG. 5B). As illustrated, the single crystals form first, and over time those crystals grow, preventing the nucleation of fused crystals. In comparing the two graphs 500, 550, the largest single crystal formed was from the mid-frequency range (line 560) and in this sample had more single crystals than fused overall in the well and with respect to the controls. The second largest single crystal sample (line 562) was the high frequency sample with the low frequency (line 564) and the controls (lines 570, 572) with the smallest.

As can be seen in the two graphs 500, 550, the mid-frequency range produces an optimal frequency such that the crystals that do nucleate and grow form a higher quality crystal. While, nucleation took longer, it resulted in higher quality crystals.

Turning now to FIG. 4, a frequency scan with respect to voltages is illustrated as a voltage phase diagram 410. In the example graph 400, only nucleation was considered and observed over a 24 hour period. At the mid-frequency, high voltage limit (area 410), no crystals were seen, as seen in the previous graph. However, at the low voltage limit (area 420), nucleation occurred. As can be seen, at the low and high frequency limits, higher voltages are required to force nucleation in these regions. The variety of crystals found over this time period is plotted in FIG. 4 where the diamonds 470 represent voltage and frequency combinations where no crystals were seen after 24 hours and the squares 480 indicate that crystals were observed after the 24 hours. It is shown that there is range of where crystallization can and cannot occur due to changes in voltages and frequency effects. It is also interesting to note that at the mid-frequency range, i.e., in the 3-5 MHz range, the crystals seen are of a different quality than those seen at low frequencies and high voltages. In particular at 20 Vpp, 300 kHz (A), the crystals in the solution were smaller and seemed to have small crystals fused together, similar to the one seen in FIG. 5A. These fused crystals were in much higher abundance in the low frequency, high voltage samples than in the high frequency high voltage samples. It can be determined from that graph 400 that frequency has an affect on how crystallization occurs and the type of crystals found in the solution. Additionally, there is shown an optimal frequency for crystallization to occur, where it requires the least amount of voltage, at, for example, 3-5 MHz. This indicates that at this frequency, the hydration cages surrounding the protein molecules in solution are disrupted.

In a second example, the electrode 120 was utilized to produce a field gradient within an example protein solution. In particular, with the verification of the frequency dependency of the quality of crystallized lysozyme and the effect of the hydration cage, the effect of the field gradient was tested to consider if the placement of the crystal formed and the size of the concentrated protein locations could be manipulated in the well. Using the example quadropole electrode 120 as illustrated in FIG. 3, an AC electric field of 8.4V and 2.917 MHz was applied for a 24 hour period and observed over 10 days. Within the first 13 hours, the control produced well formed small crystals with the biggest single crystal being roughly 56 μm large. However, the bottom of the well was scattered with small crystals that were both single and fused crystal formations. In the experimental well, there was no solid crystal formation, but, there was amorphous structures suspended in the solution that were reflecting light.

After 56 hours, the control well showed large crystals, roughly 205 μm, but they contained defects and cracks. The single crystals were still small and scattered throughout the solution. The experimental well still showed no solid nucleation but small crystals suspended in solution. After 100 hours, the control well had crystals covering the entire bottom of the well and the majority of the large crystals were cracked or had fused with adjacent crystals. As illustrated in FIG. 7, the experimental well showed large crystals 710 and minimum nucleation. The total number of nucleation sites was roughly 10 with the largest crystal measuring 215 μm. It is clear from FIG. 7 that the crystals 710 that formed are of superior quality and size. As such, with the example field gradient, the nucleation is controlled and the crystals formed are able to aid in the growth of those crystals.

Using the same voltage and frequency another well was set up with the electric field applied for only 24 hours in comparison to 3 days. The resulting crystals formed were smaller and more nucleation sites were seen. However, while crystallization appeared all over the well, no crystals were seen within a 300 μm radius from the tip of the quadropole. Along the edges of the electrodes toward the center of the well, there appeared to be a collection within the gap of the electrodes. This illustrates that prior to nucleation or when the crystals were still suspended in solution they were influenced by the strength of the field gradient. Accordingly, the crystals exhibit positive DEP properties and were attracted to the areas of high field, the electrode edges. However, because DEP is size dependent, once the crystals grew above a certain size, they crossed-over and exhibited negative DEP properties and were repelled from the high field regions. Thus the crystals congregated in the gap between the electrodes, being the low field region, or at the very center of the tip of the electrodes, or away from the electrode tips themselves. Due to the configuration of the electrodes and the applied field, the concentration is affected by AC electro-osmotic flow.

To verify that the protein crystals that formed were actual crystals, two verification techniques were used. The first was a commercial dye, (i.e., Izit Crystal Dye (Hampton Research)), that is used to differentiate between biological macromolecule crystals and inorganic crystals. The protein crystals, will allow the blue dye to penetrate into its solvent channels giving a blue appearance while the inorganic crystals will have no color effect. This process, however, only indicates that the crystals formed are protein. Accordingly, X-ray diffraction analysis was performed to verify the structures formed from the electric field are the same as those found by conventional methods. An X-ray diffraction pattern 800 shown in FIG. 8 verified that the crystal structure formed by the electric field is the same structure formed as by evaporation, thus the electric field enhances the crystallization and does not alter the way protein molecules are stretched.

Although certain example methods and apparatus have been described herein, the scope of coverage of this patent is not limited thereto. On the contrary, this patent covers all methods, apparatus and articles of manufacture fairly falling within the scope of the appended claims either literally or under the doctrine of equivalents.

We claim:

1. A method for crystallization of biomolecules comprising:
providing biomolecule solution;
bringing the biomolecule solution into direct contact with a plurality of electrodes;
applying an alternating current to the plurality of electrodes to impart a dielectrophoresis force upon the biomolecule solution; and
forming at least one crystal from the biomolecule solution.

2. A method as defined in claim 1, wherein a frequency of the alternating current is between approximately 300 kHz and approximately 18 MHz.

3. A method as defined in claim 1, wherein the plurality of electrodes are arranged substantially parallel to one another.

4. A method as defined in claim 1, wherein the plurality of electrodes are arranged in a multipole configuration.

5. A method as defined in claim 4, wherein the multipole configuration is a quadrapole.

6. A method as defined in claim 1, further comprising generating a field gradient within the biomolecule solution.

7. A method as defined in claim 6, further comprising a multipole configuration to generate the field gradient.

8. A method as defined in claim 1, wherein a voltage of the alternating current is between approximately 19.6 Vpp and approximately 21.4 Vpp.

9. A method as defined in claim 1, further comprising removing the alternating current from the plurality of electrodes after a pre-determined period of time.

10. A method as defined in claim 9, wherein the pre-determined period of time is approximately 24 hours.

11. A method as defined in claim 1, further comprising applying an alternating current to disrupt a hydration cage formed in the biomolecule solution.

12. A method as defined in claim 1, wherein the biomolecule solution is a protein solution.

13. A method as defined in claim 1, wherein a frequency of the alternating current is approximately 2.9 MHz and a voltage of the alternating current is approximately 8.4V.

14. An apparatus for the crystallization of a biomolecule comprising:
a housing to contain a biomolecule solution;
a plurality of electrodes disposed on the housing and being in direct contact with the biomolecule solution; and
an alternating current power supply electrically coupled to the plurality of electrodes to impart a dielectrophoresis force upon the biomolecule solution, the alternating current power supply having a frequency between approximately 300 kHz and approximately 18 MHz.

15. An apparatus as defined in claim 14, wherein the plurality of electrodes are arranged substantially parallel to one another.

16. An apparatus as defined in claim 14, wherein the plurality of electrodes are arranged in a multipole configuration.

17. An apparatus as defined in claim 16, wherein the multipole configuration is a quadrapole.

18. An apparatus as defined in claim 14, wherein a voltage of the alternating current is between approximately 19.6 Vpp and approximately 21.4 Vpp.
